SEQUENCE LISTING (10 sequences)

SEQ ID NO: 1 - protein, 366 aa, Rhizopus oryzae
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVHKRDIVPHVPPQSFGFLHPGVESWIKSGTSNVQICTSEIETKDCSNSIVPFTSILDHLSYFDINEGSCL

SEQ ID NO: 2 - protein, 366 aa, Artificial Sequence
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVHKRDIVPHVPSQSFGFLHPGVESWIKSGTSNVQICTSEIETKDCSNSIVPFTSILDHLSYFDINEGSCL

SEQ ID NO: 3 - protein, 366 aa, Artificial Sequence
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFRDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVHKRDIVPHVPSQSFGFLHPGVESWIKSGTSNVQICTSEIETKDCSNSIVPFTSILDHLSYFDINEGCCL

SEQ ID NO: 4 - protein, 366 aa, Artificial Sequence
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFRDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVHKRDIVPHVPPQSFGFLHPGVESWIKSGTSNVQICTSGIETKDCSNSIVPFTSILDHLSYFDINEGSCL

SEQ ID NO: 5 - protein, 366 aa, Artificial Sequence
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFRRTVHKRDIVPHVPPQSFGFLHPGVESWIKSGTSNVQICTSEIETKDCSNSIVPFTSILDHLSYFDINEGRCL

SEQ ID NO: 6 - protein, 366 aa, Artificial Sequence
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVVSTGIPFQRTVHKRDIVPHVPPQSFGFLHPGVESWIKSGTSNVQICTSEIETKDCSNSIVPFTSILDHLSYFDINEGSCL

SEQ ID NO: 7 - protein, 366 aa, Artificial Sequence
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVIDYFPVVQEQLTAHPTYKVIVTGHSLGGAQALLAGMDLYQREPRLSPKNLSIFTVGGPRVGNPTFAYYVESTGIPFQRTVHKRDTVPHVPPQSFGFLHPGVESWIKSGTSNVQICTSEIETKDCSNSIVPFTSILDHLSYFDINEGSCL

SEQ ID NO: 8 - protein, 366 aa, Artificial Sequence
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVIDYFPVVQEQLTAHPTYKVIVTGHSLGGAQALLAGMDLYQREPRLSPKNLSIFTVGGPRVGNPTFAYYVESTGISFQRTVHKRDTVPHVPPQSFGFLHPGVESWIKSGTSNVQICTSEIETKDCSNSIVPFTSILDHLSYFDINEGSCL

SEQ ID NO: 9 - protein, 366 aa, Artificial Sequence
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVHKRDIVPHVPPQSFGFLHPGVESWIKSGTSNVQICTSGIETKDCSNSIVPFTSILDHLSYFDINEGSCL

SEQ ID NO: 10 - protein, 366 aa, Artificial Sequence
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQEWVPDGKRITTFTSLLSDTNGYVLRSDKQKTIYLVFRGTNSFRSAITDIVFNFSDYKPVKGARVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVHKRDIVPHVPPLSFGFLHPGVESWIKSGTSNVQICTSEIETKDCSNSIVPFTSILDHLSYFDINEGSCL

(19) United States
(12) Patent Application Publication
(10) Pub. No.: US 2018/0355288 A1
(43) Pub. Date: Dec. 13, 2018

(54) LIPASES WITH INCREASED THERMOSTABILITY

(71) Applicant: Henkel AG & Co. KGaA, Duesseldorf (DE)

(72) Inventors: Daniela Herbst, Duesseldorf (DE); Timothy O'Connell, Landsberg am Lech (DE); Nina Mussmann, Willich (DE); Ulrich Schwaneberg, Kelmis-Hergenrath (BE); Ronny Martinez-Moya, Koeln (DE); Christian Lehmann, Biberach/Riss (DE); Volkan Besirioglu, Aachen (DE); Ljubica Vojcic, California, CA (US)

(73) Assignee: Henkel AG & Co. KGaA, Duesseldorf (DE)

(21) Appl. No.: 15/781,975

(22) PCT Filed: Nov. 23, 2016
(86) PCT No.: PCT/EP2016/078525

§ 371 Date: Jun. 6, 2018

(30) Foreign Application Priority Data
Dec. 8, 2015 (DE) 10 2015 224 576.4

(51) Int. Cl. C11D 3/386; C12N 9/20

(52) U.S. Cl. CPC C11D 3/38627; C12P 21/02; C12N 9/20

(57) ABSTRACT

The invention relates to lipases comprising an amino acid sequence having a sequence that is at least about 70% identical to the amino acid sequence specified in SEQ ID NO:1 over the entire length and an amino acid substitution of at least one of the positions K142, I149, S195, K204, N218, E287, P292, Q294, I302, P308, Q309, E335 or S364, corresponding to the numbering in accordance with SEQ ID NO:1. Such lipases have very good stability, particularly temperature stability, while providing good cleaning performance.

### CROSS-REFERENCE TO RELATED APPLICATION

This application is a U.S. National-Stage entry under 35 U.S.C. § 371 based on International Application No PCT/EP2016/078525, filed Nov. 23, 2016 which was published under PCT Article 21(2) and which claims priority to German Application No. 10 2015 224 576.4, filed Dec. 8, 2015, which are all hereby incorporated in their entirety by reference.

### TECHNICAL FIELD

The present disclosure relates to enzyme technology. The disclosure relates to lipases of rhizopus oryzae whose amino acid sequence has been changed in order to gain better thermal stability, particularly in regard to use in detergents and cleaning agents, and the coding nucleic acids for them, as well as the production thereof. The disclosure also relates to uses of these lipases and methods in which they are used, as well as agents containing said lipases, particularly detergents and cleaning agents.

### BACKGROUND

Lipases are among the most technically important enzymes. Their use in detergents and cleaning is established industrially and they are contained in practically all modern, high-performance detergents and cleaning agents. Lipases are enzymes which catalyze the hydrolysis of ester bonds in lipid substrates, particularly in greases and oils, and thus are a part of the group of esterases. Lipases are typically enzymes which can cleave a plurality of substrates, e.g. aliphatic, alicyclic, bicyclic and aromatic esters, thioesters and activated amines. Lipases are used to remove fatty stains by catalyzing their hydrolysis (lipolysis).

Lipases with broad substrate spectra are used, in particular, where heterogeneous raw materials or substrate mixtures must be converted, such as in detergents and cleaning agents, because stains can consist fats and oils having different compositions. The lipases in the detergents or cleaning agents known from the prior art normally have a microbial origin and originate from bacteria or fungus, particularly the species bacillus, pseudomonas, acinetobacter, micrococcus, humicola, trichoderma or trichosporon. Lipases are normally produced according to known biotechnological methods with suitable microorganisms, such as by means of transgenic expression host of the species bacillus or by filamentous fungi.

The European patent application EP 0443063, for example, discloses a lipase of pseudomonas sp. ATCC 21808 provided for a detergent and cleaning agent. Japanese patent application JP 1225490 discloses a lipase of rhizopus oryzae. In general, only select lipases are suitable for use in liquid preparations containing surfactants. Many lipases do not have an adequate catalytic effect or stability in such preparations. In washing methods which are generally carried out at temperatures above 20° C., in particular, many lipases are thermally unstable, which results in inadequate catalytic activity during the washing process. In liquid surfactant preparations containing phosphonates, this problem is more serious, i.e. due to the complexing properties of the phosphonates or due to the unfavorable interactions between the phosophonate and the lipases.

Consequently, liquid formulations from the prior art containing lipases and surfactants have the disadvantage that they frequently do not have satisfactory lipolytic activity in the temperature ranges which a washing method requires and thus do not have optimal cleaning performance on lipase-sensitive stains.

### BRIEF SUMMARY

A lipase is provided herein. The lipase includes an amino acid sequence having a sequence that is at least about 70% identical to the amino acid sequence specified in SEQ ID NO:1 over the entire length. The amino acid sequence has an amino acid substitution of at least one of the positions K142, I149, S195, K204, N218, E287, P292, Q294, I302, P308, Q309, E335 or S364, corresponding to the numbering in accordance with SEQ ID NO:1.

A method for production of a lipase is also provided herein. The lipase includes the substitution of an amino acid in at least one of the positions corresponding to position 142, 149, 195, 204, 218, 287, 292, 294, 302, 308, 309, 335 and 364 in SEQ ID NO:1, in an original lipase having a sequence that is at least about 70% identical to the amino acid sequence specified in SEQ ID NO:1 over the entire length such that the lipase includes the amino acid substitution K142E, I149R, S195R, K204R, N218I, E287V, P292S, Q294R, I302T, P308S, Q309L, E335G, S364C or S364R in at least one position.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, it has been found that a lipase of rhizopus oryzae or an adequately similar lipase (in relation to the sequence identity), which has an amino acid substitution of at least one of the positions K142, I149, S195, K204, N218, E287, P292, Q294, I302, P308, Q309, E335 or S364, corresponding to the numbering according to SEQ ID NO:1, is improved with respect to (thermal) stability in comparison with the wild type form and is thus particularly well-suited for use in detergents and cleaning agents.

The subject as contemplated herein in a first aspect, therefore, is a lipase comprising an amino acid sequence having a sequence that is at least about 70% identical to the amino acid sequence specified in SEQ ID NO:1 over the entire length and an amino acid substitution of at least one of the positions K142, I149, S195, K204, N218, E287, P292, Q294, I302, P308, Q309, E335 or S364, corresponding to the numbering in accordance with SEQ ID NO:1.

An additional subject as contemplated herein is a method for production of a lipase comprising the substitution of an amino acid in at least one of the positions corresponding to position 142, 149, 195, 204, 218, 287, 292, 294, 302, 308, 309, 335 or 364 in SEQ ID NO:1, in an initial lipase having a sequence that is at least about 70% identical to the amino acid sequence specified in SEQ ID NO:1 over the entire length such that the lipase has at least one of the amino acid substitutions K142E, I149R, S195R, K204R, N218I, E287V, P292S, Q294R, I302T, P308S, Q309L, E335G, S364C or S364R.

A lipase in the context of the disclosure, therefore, comprises both the lipase as such and a lipase produced with a method as contemplated herein. All embodiments for the lipase thus relate to the lipase itself and such lipases which are produced by means of the corresponding methods.

Additional aspects as contemplated herein relate to the coding nucleic acids for these lipases, lipases as contemplated herein or nucleic acids containing non-human host cells, as well as agents as contemplated herein comprising lipases, particularly detergents and cleaning agents, washing and cleaning methods and uses of the lipases in detergents or cleaning agents for removal of fatty stains.

In this case, “at least one” is understood to mean one or multiple, i.e. 1, 2, 3, 4, 5, 6, 7, 8, 9, 10, 11, 12, 13, 14 or more.

The present disclosure is based on the surprising result that an amino acid substitution of at least one of positions 142, 149, 195, 204, 218, 287, 292, 294, 302, 308, 309, 335 or 364 of the lipase of rhizopus oryzae according to SEQ ID NO:1 in a lipase which comprises an amino acid sequence that is at least about 70% identical to the amino acid sequence specified in SEQ ID NO:1 such that the amino acids 142E, 149R, 195R, 204R, 218I, 287V, 292S, 294R, 302T, 308S, 309L, 335G, 364C or 364R are present in at least one of the corresponding positions achieves improved (thermal) stability of the modified lipase in detergents and cleaning agents. This particularly surprising to the extent that none of the aforementioned amino acid substitutions were previously associated with increased stability of the lipase.

The lipases have increased stability in detergents and cleaning agents, particularly in relation to increased temperatures. Such enhanced lipases enable improved washing results on lipolitically sensitive stains in a wide temperature range.

The lipases have enzymatic activity, which means they are capable of hydrolysis of fats and oils, particularly in a detergent or cleaning agent. A lipase as contemplated herein, therefore, is an enzyme which catalyzes the hydrolysis of ester bonds in liquid substrates and is thus capable of cleaving fats or oils. Moreover, the lipase is preferably a mature lipase, i.e. a catalytically active module without signals and/or propeptides. Unless specified otherwise, the indicated sequence relates to mature (processed) enzymes.

In various embodiments, the lipase includes at least one amino acid substitution selected from the group of K142E, I149R, S195R, K204R, N218I, E287V, P292S, Q294R, I302T, P308S, Q309L, E335G, S364C and S364R, corresponding to the numbering in accordance with SEQ ID NO:1. In other preferred embodiments, the lipase contains one of the following amino acid substitution variants: (i) P308S; (ii) S195R and S364C; (iii) S195R and E335G; (iv) Q294R and S364R; (v) E287; (vi) N218I and I302T; (vii) P292S; (viii) E335G; or (ix) K142E, I149R, K204R and Q309L, where the numbering is corresponding to the numbering according to SEQ ID NO:1.

In another preferred embodiment as contemplated herein, the lipase contains an amino acid sequence which is at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5% and about 98.8% is identical to the amino acid sequence specified in SEQ ID NO:1 over the entire length, and which has one ore multiple of the amino acid substitutions 142E, 149R, 195R, 204R, 218I, 287V, 292S, 294R, 302T, 308S, 309L, 335G, 364C or 364R in at least one of the positions 142, 149, 195, 204, 218, 287, 292, 294, 302, 308, 309, 335 or 364 in the count according to SEQ ID NO. 1. In the context of the present disclosure, this means the feature that a lipase which has the specified substitutions, that at least one of the corresponding amino acids is contained at the corresponding position, i.e. not all of the 14 positions must be mutated or deleted, for example, by means of fragmentation of the lipase. The amino acid sequences of such lipases which are preferred as contemplated herein are specified in SEQ ID Nos: 2-10.

Determination of the identity of nucleic acid or amino acid sequences takes place with a sequence comparison. This sequence comparison is based on the BLAST algorithm established in the prior art and which is normally used (see Altschul, S. F., Gish, W., Miller, W., Myers, E. W. & Lipman, D. J. (1990) “Basic local alignment search tool.” J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs”; Nucleic Acids Res., 25, S.3389-3402) and essentially takes place in a manner such that similar sequences of nucleotides or amino acids in the nucleic acid sequences are assigned to each other. A tabular assignment of the relevant positions is referred to as alignment. An additional algorithm available in the prior is the FASTA algorithm. Sequence comparisons (alignments), particularly multiple sequence comparisons, are created with computer programs. For example, the Clustal series (refer, for example, to Chenna et al. (2003): Multiple sequence alignment with the Clustal series of programs. Nucleic Acid Research 31, 3497-3500), T-Coffee (refer, for example, to Notredame et al. (2000): T-Coffee: A novel method for multiple sequence alignments. J. Mol. Biol. 302, 205-217) or programs based on these programs or algorithms are used. Sequence comparisons (alignments) with the computer program Vector NTI® Suite 10.3 (Invitrogen Corporation, 1600 Faraday Avenue, Carlsbad, Calif., USA) with the specified standard parameters whose AlignX module for the sequence comparisons is based on ClustalW are also possible. Unless specified otherwise, the sequence identified indicated here is determined with the BLAST algorithm.

Such a comparison also permits a statement about the similarity of the compared sequences to each other. They are normally specified in percent identity, which means the portion of identical nucleotides or amino acid radicals on the same positions or in positions corresponding to each other in an alignment. The additional encompassed term of homology relates to preserved amino acid substitutions in consideration with amino acid sequences, i.e amino acids having similar chemical activity, since they usually exert similar chemical activities within the protein. Therefore, the similarity of the comparable sequences can also specify percent homology or percent similarity. Identity and/or homology specifications can apply over the complete polypeptide or gene or only individual ranges. Homology or identical ranges of various nucleic acid or amino acid sequences are thus defined by matches in the sequences. Such ranges often have identical functions. They can be small and comprise only a few nucleotides or amino acids. Such small ranges often perform essential functions for the overall activity of the protein. Therefore, it can be beneficial to relate sequential matches to only individual or small ranges. However, if nothing different is indicated, identity or homology specifications in the present application relate to the total length of the respective specified nucleic acid or amino acid sequence.

In the context of the present disclosure, therefore, the specification that an amino acid position corresponds to a numerically identified position in SEQ ID NO:1, so the corresponding position of the numerically identified position in SEQ ID NO:1 is assigned in an alignment as defined above.

In a further embodiment as contemplated herein, the lipase is wherein its cleaning performance is not significantly reduced in comparison with that of a lipase comprising an amino acid sequence corresponding to the amino acid sequence specified in SEQ ID NO:1, which is to say that it retains at least about 80% of the reference washing performance, preferably at least about 100%, more preferably at least about 110%. The cleaning performance can be determined in a washing system that contains a detergent in a dosage between from about 4.5 and about 7.0 grams per liter of washing liquor, wherein the lipases to be compared are used in an equal concentration (relative to active protein) and the cleaning performance on a stain on cotton is determined by measuring the cleaning degree of washed textiles. For example, the washing process can take place for about 70 minutes at a temperature of about 40° C. and the water has a hardness between from about 15.5 and about 16.5° (German hardness). The concentration of lipases in the detergent intended for this washing system is from about 0.001 to about 0.1 wt. %, preferably from about 0.01 to about 0.06 wt. %, relative to active, cleaned protein.

A liquid reference detergent for such a washing system can be composed as follows (all specifications in percentage by weight): about 7% alkylbenzene sulfonic acid, about 9% further anionic surfactants, about 4% C12-C18 Na-salts of fatty acids (soaps), about 7% nonionic surfactants, about 0.7% phosphonates, about 3.2% citric acid, about 3.0% NaOH, about 0.04% defoamer, about 5.7% 1,2-propanediol, about 0.1% preservative, about 2% ethanol, about 0.2% dye-transfer inhibitor, residual demineralized water. The dosage of the liquid detergent is preferably between from about 4.5 and about 6.0 grams per liter of washing liquor, e.g. about 4.7, about 4.9 or about 5.9 grams per liter of washing liquor. Washing preferably takes place in a pH value range between about pH 8 and about pH 10.5, preferably between about pH 8 and about pH 9.

In the context of the present disclosure, the determination of the cleaning performance takes place, for example, at 34.8° C. using a liquid detergent as specified above, wherein the washing process preferably takes place for 30 minutes.

The degree of whiteness, i.e. the lightening of stains, is determined in an optical measuring process, preferably photometric, as a measure for cleaning performance. A suitable device for this purpose, for example is the Minolta CM508d spectrometer. Normally, the devices used for the measurement are pre-calibrated with a white standard, preferably a white standard supplied with the device.

With the activity-equivalent use of the respective lipase, it is ensured that the respective enzymatic properties, i.e. the cleaning performance on certain stains, are also compared with any divergence of the behavior of active substance from the overall protein (the values of the specific activity). In general, a low specific activity can be compensated by adding a larger amount of protein.

Otherwise, the lipase activity can also be determined in a manner familiar to a person skilled in the art, preferably as described in Bruno Stellmach, “Bestimmungsmethoden Enzyme fur Pharmazie, Lebensmittelchemie, Technik, Biochemie, Biologie, Medizin [Methods for determining enzymes for pharmacy, food chemistry, technology, biochemistry, medicine]” (Steinkopff Verlag Darmstadt, 1988, p. 172ff). In the process, samples containing lipases are added to an olive oil emulsion in water containing emulsifier and incubated at 30° C. and pH 9.0. The fatty acids are released in the process. They are titrated with an autotitrator for 20 minutes with a 0.01 N caustic soda so that the pH value remains constant (“pH-stat-titration”). The lipase activity is determined based on the caustic soda consumption in relation to a reference lipase sample.

An alternative test for determining the lipolytic activity of the lipases is an optical measuring method, preferably a photometric method. The test suitable for this purpose comprises the lipase-dependent cleavage of the substrate para-nitrophenol-butyrate (pNP-butyrate). This is cleaved by the lipase in para-nitrophenolate and butyrate. The presence of para-nitrophenolate can be determined by using a photometer, e.g. the Tecan Sunrise device and the XFLUOR software, at 405 nm and thus enables conclusions about the enzymatic activity of the lipase.

The protein concentration can be determined with known methods, e.g. the BCA method (bicinchoninic acid; 2,2′-biquinolyl-4,4′-dicarboxylic acid) or the biuret method (A. G. Gornall, C. S. Bardawill and M. M. David, J. Biol. Chem., 177 (1948), p. 751-766). Determination of the active protein concentration in this respect can take place with titration of the active centers using a suitable irreversible inhibitor and determination of the residual activity cf. M. Bender et al., J. Am. Chem. Soc. 88, 24 (1966), p. 5890-5913).

In addition to the amino acid changes explained above, lipases can have additional amino acid changes, particularly amino acid substitutions, insertions or deletions. Such lipases are, for example, enhanced with a purposeful genetic change, i.e. mutagenic methods, and optimized for specific applications or with respect to specific properties (e.g. in regard to their catalytic activity, stability, etc.). Moreover, nucleic acids can be added in recombination approaches and the used to produce completely new lipases or other polypeptides.

The goal is to introduce intentional mutations to the known molecule, such as substitutions, insertions or deletions, in order to improve the cleaning performance of enzymes. In particular, the surface charges and/or isoelectric point of the molecule and thus their interactions with the substrate can be changed for this purpose. Therefore, the net charge of the enzyme, for example, can be changed in order to influence the substrate binding, particularly for use in detergents and cleaning agents. Alternatively or additionally, the stability of the lipase can be further increased and thus the cleaning performance improved by one or multiple corresponding mutations. Advantageous properties of individual mutations, e.g. individual substitutions, can be supplemented. Therefore, a lipase already optimized in regard to specific properties, e.g. in respect to its stability under increased temperatures, can also be enhanced in the scope as contemplated herein.

For the description of substitutions which relate to exactly one amino acid position (amino acid exchanges), the following convention is applied here: first, the naturally existing amino acid is identified in the form of the conventional international single-letter code, then the corresponding sequential position and finally the inserted amino acid. Multiple exchanges within the same polypeptide chain are separated by slashes. With insertions, additional amino acids are named according to the sequential position. With deletions, the missing amino acid is replaced by a symbol, such as a start or a dash, or indicated by a A before the corresponding position. For example, K142E describes the substitution of lysine at position 142 with glutamic acid, K142KE describes the insertion of glutamic acid after the amino acid lysine at position 142 and K142* and AK142 describes the deletion of lysine at position 142. This nomenclature is known to a person skilled in the art in the field of enzyme technology.

A further subject as contemplated herein, therefore, is a lipase wherein it is obtained from a lipase as described above as an initial molecule with single or multiple conservative amino acid substitution, wherein the lipase in the sequence according to SEQ ID NO:1 still has at least one of the amino acid substitutions at the positions corresponding to the positions 142, 149, 195, 204, 218, 287, 292, 294, 302, 308, 309, 335 and 364 in SEQ ID NO:1, as described above. The term “conservative amino acid substitution” is understood to means the replacement (substitution) of an amino acid radical with a different amino acid radical, wherein this substitution does not cause a change in the polarity or charge at the position of the replaced amino acid, i.e. the replacement of a non-polar amino acid radical with a different non-polar amino acid radical. Conservative amino acid substitutions in the context of the disclosure comprise, for example: G=A=S, I=V=L=M, D=E, N=Q, K=R, Y=F, S=T, G=A=I=V=L=M=Y=F=W=P=T.

Alternatively or supplementary, the lipase is wherein it can be obtained from a lipase as contemplated herein by fragmenting, deletion-, insertion- or substitution mutagenesis and comprises an amino acid sequence which matches the original molecule over a length of at least about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 361, about 362, about 363, about 364 or about 365 linked amino acids, wherein the amino acid substitution(s) contained in the original molecule is/are still present at one or multiple positions corresponding to the positions 142, 149, 195, 204, 218, 287, 292, 294, 302, 308, 309, 335 and 364 in SEQ ID NO:1.

Thus, for example, it is possible to delete individual amino acids at the termini in the loops of the enzyme without the lipolytic activity being lost or reduced as a result. Moreover, the allergenicity of relevant enzymes can be reduced by means of such fragmenting, deletion-, insertion- or substitution mutagenesis and thus their overall usability can be improved. The enzymes also beneficially retain their lipolytic activity after the mutagenesis, i.e. their lipolytic activity corresponds to at least that of the original enzyme, i.e in a preferred embodiment, the lipolytic activity is at least about 80, preferably at least about 90% of the activity of the original enzyme. Additional substitutions can also have beneficial effects. Individual and multiple linked amino acids can be substituted with other amino acids.

Alternatively or supplementally, the lipase is wherein it can be obtained from an lipase as an original molecule by means of single or multiple conservative amino acid substitution, wherein the lipase has at least one of the amino acid substitutions K142E, I149R, S195R, K204R, N218I, E287V, P292S, Q294R, I302T, P308S, Q309L, E335G, S364C or S364R, corresponding to the positions 142, 149, 195, 204, 218, 287, 292, 294, 302, 308, 309, 335 and 364 according to SEQ ID NO:1.

In further embodiments, the lipase is wherein it can be obtained from a lipase as contemplated herein by fragmenting, deletion-, insertion- or substitution mutagenesis and comprises an amino acid sequence which matches the original molecule over a length of at least about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360 or about 366 linked amino acids, wherein the lipase comprises at least one of the amino acid substitutions K142E, I149R, S195R, K204R, N218I, E287V, P292S, Q294R, I302T, P308S, Q309L, E335G, S364C or S364R corresponding to the positions 142, 149, 195, 204, 218, 287, 292, 294, 302, 308, 309, 335 and 364 according SEQ ID NO:1.

The additional amino acid positions are defined here by an alignment of the amino acid sequence of a lipase with the amino acid sequence of the lipase of rhizopus oryzae, as specified in SEQ ID NO:1. Furthermore, the assignment of positions is based on the mature protein. This assignment must also be applied, in particular, when the amino acid sequence of a lipase comprises a higher number of amino acid radicals than the lipase of rhizopus oryzae according to SEQ ID NO. 1. Starting from the indicated positions in the amino acid sequence of the lipase of rhizopus oryzae, the change positions in a lipase are those which are assigned to these positions in an alignment.

Advantageous positions for sequence changes, particularly substitutions, of the lipase of rhizopus oryzae, which are preferably important to homologous positions of the lipases and give the lipase beneficial functional properties are, accordingly, the positions corresponding to the positions 142, 149, 195, 204, 218, 287, 292, 294, 302, 308, 309, 335 and 364 in SEQ ID NO:1 in an alignment, i.e. in the sequence according to SEQ ID NO:1. The following amino acid radicals are present at the indicated positions in the wild type molecule of the lipase of rhizopus oryzae: K142, I149, S195, K204, N218, E287, P292, Q294, I302, P308, Q309, E335 and S364.

Comparison tests can provide additional confirmation of the correct assignment of the amino acids to be changed, i.e. particularly their functional equivalent, according to which both assigned positions are changed in the same manner in both lipases compared with each other on the basis of an alignment and it can be observed whether the enzymatic activity is changed in the same manner for both. If, for example, an amino acid exchange is included at a specific position of the lipase of rhizopus oryzae according to SEQ ID NO:1 with a change of an enzymatic parameter, for example with an increase of the KM value, and if a corresponding change of the enzymatic parameter, for example an increase of the KM value, is observed in a lipase variant whose amino acid substitution was achieved with introduction of the same amino acid, the correct assignment is confirmed.

All indicated circumstances are also applicable to the method for production of a lipase. Accordingly, a method also comprises one or more of the following method steps:
a) Introduction of a single or multiple conservative amino acid substitution, wherein the lipase comprises at least one of the amino acid substitutions K142E, I149R, S195R, K204R, N218I, E287V, P292S, Q294R, I302T, P308S, Q309L, E335G, S364C or S364R, corresponding to the positions 142, 149, 195, 204, 218, 287, 292, 294, 302, 308, 309, 335 and 364 according to SEQ ID NO:1.
b) Change of the amino acid sequence by fragmenting, deletion-, insertion- or substitution mutagenesis such that the lipase comprises an amino acid sequence which matches the original molecule over a length of at least about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360 or about 366 linked amino acids, wherein the lipase comprises at least one of the amino acid substitutions K142E, I149R, S195R, K204R, N218I, E287V, P292S, Q294R, I302T, P308S, Q309L, E335G, S364C or S364R corresponding to the positions 142, 149, 195, 204, 218, 287, 292, 294, 302, 308, 309, 335 and 364 according SEQ ID NO:1.

All embodiments also apply for the methods.

In additional variants as contemplated herein, the lipase or the lipase produced with the method as contemplated herein is still at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5% or about 98.8% identical to the amino acid sequence specified in SEQ ID NO:1 over the entire length. Alternatively, the lipase or the lipase produced with the method as contemplated herein is still at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5% or about 98%, identical to one of the amino acid sequences specified in SEQ ID Nos:2-10 over the entire length. The lipase or the lipase produced with the method as contemplated herein has an amino acid substitution in at least one of the positions K142, I149, S195, K204, N218, E287, P292, Q294, I302, P308, Q309, E335 or S364, corresponding to the numbering according to SEQ ID NO:1 in each case. In more preferred embodiments, the amino acid substitution is at least one substitution selected from the group of K142E, I149R, S195R, K204R, N218I, E287V, P292S, Q294R, I302T, P308S, Q309L, E335G, S364C and S364R, corresponding to the numbering in accordance with SEQ ID NO:1. In other preferred embodiments, the lipase comprises one of the following amino acid substitution variants: (i) P308S; (ii) S195R and S364C; (iii) S195R and E335G; (iv) Q294R and S364R; (v) E287; (vi) N218I and I302T; (vii) P292S; (viii) E335G; or (ix) K142E, I149R, K204R and Q309L.

An additional subject as contemplated herein is a lipase as described above, which is also stabilized, in particular, by one or multiple mutations, such as substitutions, or by coupling to a polymer. An increase of the stability during storage and/or during use, e.g. during the washing process, entails that the enzymatic activity lasts longer and thus the cleaning performance is improved. Basically, all stabilization possibilities described in the prior art and/or which are purposeful come into consideration. Preference is given to such stabilizations which are achieved with mutations of the enzyme itself, because such stabilizations do not require additional work steps after the enzyme is procured. examples for suitable sequence change for this purpose are indicated above. Additional suitable sequence changes are known from the prior art.

Possibilities of stabilization are, for example:

Protection from the influence of denatured agents, such as surfactants, by mutations which cause a change to the amino acid sequence on the surface of the protein;

exchange of amino acids, which lie close to the N-terminus, against those, which presumably come into contact with the rest of the molecule via non-covalent interactions and thus contribute to the maintenance of the globular structure.

Preferred embodiments are such embodiments in which the enzyme is stabilized in multiple ways, because stabilizing mutations have an additive or synergistic effect.

An additional subject as contemplated herein is a lipase as described above, wherein it has at least one chemical modification. A lipase with such a change is called a derivative, i.e. the lipase is derivatized.

In the context of the present disclosure, derivatives are understood to mean such proteins whose amino acid chain has been chemically modified. Such derivatizations can, for example, take place in vivo with the host cell, which expresses the protein. Couplings of low-molecule compounds, such as compounds of lipids or oligosaccharides, should be emphasized, in particular. Derivatizations can also be carried out in vitro, e.g. by chemical conversion of a side chain of an amino acid or by covalent bonding of an additional compound to the protein. For example, the coupling of amines to carboxyl groups of an enzyme to change the isoelectric point is possible. Another such compound can also be an additional protein, which, for example, is bound to a protein by bifunctional chemical compounds. Derivatization is also understood to mean the covalent bonding on a macromolecular carrier or a non-covalent inclusion in suitable macromolecular cage structures. Derivatizations can, for example, influence substrate specificity or the bonding strength on the substrate or cause a temporary blocking of enzymatic activity when the coupled substance is an inhibitor. This can be beneficial, for example, for a period of storage. Moreover, such modifications can influence the stability or enzymatic activity. They can also reduce the allergenicity and/or immunogenicity of the protein and thus increase its skin tolerance. For example, couplings with macromolecular compounds, such as polyethylene glycol, improve the protein in regards to its stability and/or skin tolerance.

Derivatives of a protein as contemplated herein can also be understood to mean preparations of said proteins in the broadest sense. Depending on the procurement, processing or preparation, a protein can be combined with various other substances, i.e. from the culture of the producing microorganisms. A protein can also have been purposely supplemented with additional substances to, for example, increase its storage stability. Therefore, all preparations of a protein as contemplated herein are also inventive. This is also independent of whether this enzymatic activity actually develops in a specific protein or not. It may be desired that there is little or no activity during storage, and the enzymatic function does not develop until the time of use. This can be controlled, for example, with appropriate concomitant substances. In particular, the common preparation of lipases with specific inhibitors is possible in this respect.

Among all of the lipases or lipase variants and/or derivatives described above, particular preference in the context of the present disclosure is given to those whose stability and/or activity corresponds to that of the lipases according to SEQ ID Nos: 2-10, and/or whose cleaning performance corresponds to that of at least one of the lipases according SEQ ID Nos: 2-10, wherein the cleaning performance in a washing system is determined as described above.

An additional subject of the present disclosure is a nucleic acid which is coded for a lipase as contemplated herein, as well as a vector containing one such nucleic acid, particularly a cloning vector or an expression vector.

This can be a DNA or RNA molecule. It can also be present as a single strand, as a single strand complementary to said single strand, or as a double strand. With DNA molecules, in particular, the sequences of both complementary strands must each be taken into consideration in all three possible reading frames. Moreover, it must be taken into consideration that various codons, i.e. base triplets, can be coded for the same amino acids, so that a specific amino acid sequence of multiple different nucleic acids can be codes. Due to this degeneracy of the genetic code, all nucleic acid sequences which can encode one of the lipases described above are included in this subject matter as contemplated herein. A person skilled in the art is able to determine these nucleic acid sequences beyond doubt, because individual codons are assigned to defined amino acids despites the degeneracy of the genetic code. Therefore, a person skilled in the art can, based on an amino acid sequence, easily determine coding nucleic acid for this amino acid sequence. Furthermore, one or multiple codons can be replaced with synonymous codons in nucleic acids as contemplated herein. This aspect relates, in particular, to the heterological expression of the enzymes. Therefore, each organism, such as a host cell of a production strain, has a specific codon use. The term codon us is understood to mean the translation of the genetic code to amino acids by the respective organism. Bottlenecks in the protein biosynthesis can occur of codons on the nucleic acid in the organism have a comparatively lower number of charged tRNA molecules. Although coding takes place for the same amino acid this has the effect that a codon is translated less efficiently in the organism than a synonymous for the same amino acid. Due to the presence of a higher number of tRNA molecules for the synonymous codon, this can be translated more efficiently in the organism.

A person skilled in the art can apply generally known methods, such as chemical syntheses or polymerase chain reaction (PCR) in combination with standard molecular-biological and/or protein-chemical methods to produce the appropriate nucleic acids up to complete genes on the basis of known DNA and/or amino acid sequences. Such methods are known, for example, from Sambrook, J., Fritsch, E. F. and Maniatis, T. 2001. Molecular cloning: a laboratory manual, 3. Edition Cold Spring Laboratory Press.

In the context of the present disclosure, vectors are understood to mean elements consisting of nucleic acids which contain a nucleic acid as an identifying nucleic acid range. They may establish this in a species or cell line over multiple generations or cell divisions as a stable genetic element. Vectors are, particularly when used in bacteria, special plasmids, i.e. circular genetic elements. A nucleic acid is cloned to a vector in the scope of the present disclosure. The vectors include, for example, those whose origin is bacterial plasmids, viruses or bacteriophages, or predominantly synthetic vectors or plasmids with elements of different origins. With the additional present genetic elements, vectors can be established as a stable unit in the relevant host cells over multiple generations. The can be present as extrachromosomal units or integrate into a chromosome or chromosomal DNA.

Expression vectors comprise nucleic acid sequences which enable them to be introduced into the host cells containing them, preferably microorganisms, particularly bacteria, and to bring a nucleic acid contained therein to expression. The expression is influence, in particular, by the promotor or promotors which regulate the transcription. Basically, the expression can take place with the natural, original promotor localized before the nucleic acid to be expressed, but also by a promotor of the host cell prepared on the expression vector or by a modified or a completely different promotor of another organism or another host cell. In the present case, at least one promotor is available for the expression of a nucleic acid as contemplated herein and used for the expression thereof. Moreover, expression vectors can be regulated, for example, by changing the cultivation conditions or when reaching a specific cell density of the host cells contained therein or with addition of specific substances, particularly activators of the gene expression. An example of one such substance is the galactose derivative isopropyl-β-thiogalactopyranosid (IPTG), which is used as an activator of the bacterial lactose operon (lac operon). Unlike express vectors, the contained nucleic acids are not expressed in cloning vectors.

An additional subject as contemplated herein is a non-human host cell which contains a nucleic acid or a vector, or which contains a lipase, particularly one which secretes the lipase into the medium surrounding the host cell. Preferably, a nucleic acid as contemplated herein or a vector as contemplated herein is transformed into a microorganism which then represents a host cell as contemplated herein. Alternatively, individual components, i.e. nucleic acid parts or fragments of a nucleic acid as contemplated herein, can be introduced to a host cell so that the resulting host cell contains a nucleic acid as contemplated herein or a vector as contemplated herein. This procedure is ideally suited when the host cell already contains one or multiple components of a nucleic acid or vector and the other components are then supplemented accordingly. Methods for transformation of cells are established in the prior art and have been known to the person skilled in the art for a long time. Basically, all cells, i.e. prokaryotic or eukaryotic cells, are suitable as host cells. Preference is given to such host cells which can be beneficially handled genetically, which applies, for example, to transformation with the nucleic acid or the vector and its stable establishment, such as single-cell fungi or bacteria. Moreover, preferred host cells are characterized by a good microbiological and biotechnical manageability. This relates, for example, the ability to cultivate easily, high growth rates, low demands on fermentation media and good production and secretion rates for foreign proteins. Preferred host cells as contemplated herein secrete the (transgenic) expressed protein into the medium surround the host cells.

Moreover, the lipases can be modified by the cells producing them after their preparation, for example by linking sugar molecules, formulations, aminations, etc., post-translational modifications can functionally influence the lipase.

Additional preferred embodiments are such host cells which can be regulated in their activity on the basis of genetic regulation elements provided, for example, on the vector, but can also be present in these cells beforehand. For instance, they can be brought to expression with controlled addition of chemical compounds which serve as activators, by changing the cultivation conditions or upon reaching a specific cell density. This enables efficient production of the proteins. An example of such a compound is IPTG, as described above.

Preferred host cells are prokaryotic or bacterial cells. Bacteria are characterized by short generation times and low demands on cultivation conditions. Consequently, cost-effective cultivation methods or production methods can be established. The person skilled in the art can also refer to extensive experience with bacteria in fermentation technology. For special production, it is possible to use a wide variety of bases, such as nutrient sources, product formation rate, time requirement, etc., gram-negative or gram-positive bacteria, which can be determined experimentally in the individual case.

With gram-negative bacteria, such as escherichia coli, a plurality of proteins is secreted to the periplasmatic space, i.e. into the compartment between the membranes containing the two cells. This can be beneficial for special applications. Furthermore, gram-negative bacteria can also be designed in such a way that they penetrate the expressed proteins in the periplasmatic space and into the medium surrounding the bacterium. Gram-positive bacteria, such as bacilli or actinomycetes or other representatives of actinomycetales have no outer membrane, so that secreted proteins are immediately introduced into the medium surrounding the bacteria, usually the nutrient medium, from which the expressed proteins can be purified. They can be isolated directly from the medium or further processed.

In addition, gram-positive bacteria are related or identical to most of the origin organisms for industrially important enzymes and usually form comparable enzymes, so that they have a similar codon usage and their protein synthesis apparatus is naturally oriented accordingly.

Host cells as contemplated herein can be changed with respect to their demands on the culture conditions, have different or additional selection markers or express different or additional proteins. In particularly, they may be such host cells which express multiple proteins or enzymes.

The present disclosure is basically applicable to all microorganisms, particularly all fermentable microorganisms, preferably those of the species bacillus, and enable production of proteins as contemplated herein with the use of such microorganisms. Such microorganisms then represent host cells in the context of the disclosure.

In a further embodiment as contemplated herein, the host cell is wherein it is a bacterium, preferably one, which is selected from the group of the species of escherichia, klebsiella, bacillus, staphylococcus, corynebacterium, arthrobacter, streptomyces, stenotrophomonas and pseudomonas, more preferably one, which is selected from the group of escherichia coli, klebsiella planticola, bacillus licheniformis, bacillus lentus, bacillus amyloliquefaciens, bacillus subtilis, bacillus alcalophilus, bacillus globigii, bacillus gibsonii, bacillus clausii, bacillus halodurans, bacillus pumilus, staphylococcus carnosus, corynebacterium glutamicum, arthrobacter oxidant, streptomyces lividans, streptomyces coelicolor and stenotrophomonas malphilia.

The host cell can also be a eukaryotic cell wherein it has a cell nucleus. Therefore, a further subject as contemplated herein is a host cell wherein it has a cell nucleus. Unlike prokaryotic cells, eukaryotic cells are capable of modifying the formed protein post-translationally. Examples of this are fungi such as actinomycetes or yeasts such as saccharomyces or kluyveromyces. This can be particularly advantage when, for example, the proteins should undergo specific modifications in connection with their synthesis in order to enable such systems. The modifications which eukaryotic systems carryout in connection with protein synthesis, in particular, include, for example, the bonding of low-molecular compounds, such as membrane anchors or oligosaccharides. Such oligosaccharide modifications can be desired to, for example, reduce the allergenicity of an expressed protein. Co-expression with the enzymes naturally formed by such cells, such as cellulases, can be beneficial. Moreover, thermophilic fungal expression systems can be particularly well-suited for expression of temperature-resistant proteins or variants.

The host cells as contemplated herein are cultivated in the usual manner and fermented, for example, in discontinuous or continuous systems. In the first case, a suitable nutrient medium is innoculated with the host cells and the product is harvested from the medium after a time determined experimentally. Continuous fermentations are characterized by the achievement of a flow equilibrium in which cells partly die over a comparatively long time but also multiply and the formed protein can be simultaneously extracted from the medium.

Host cells as contemplated herein are preferably used in order to produce lipases as contemplated herein. An additional subject as contemplated herein, therefore, is a method for production of a lipase comprising
a) cultivation of a host cell, and
b) isolation of the lipase from the culture medium or the host cell.

This subject as contemplated herein preferably comprises fermentation processes. Fermentation processes are known from the prior art and are actually the most technical production step, normally followed by a suitable purification method of the product produced, for example of the lipases. All fermentation processes based on a corresponding method for production of a lipase as contemplated herein are embodiments of this subject matter as contemplated herein.

Fermentation processes wherein the fermentation is carried out by means of an inflow strategy, in particular, come into consideration. In this connection, the media components which are consumed by the ongoing cultivation, are continuously fed. Consequently, considerable increases in both cell density and cell mass and/or dry mass and/or in the activity of the interesting lipase, in particular, are achieved. Moreover, the fermentation can also be designed so that undesired metabolic products can be filtered out or neutralized with the addition of buffers or suitable counterions.

The produced lipase can be harvested from the fermentation medium. Such a fermentation process is preferable to an isolation of the lipase from the host cell, i.e. a production preparation from the cell mass (dry mass), however, the provision of suitable host cells or of one or multiple suitable secretion markers or mechanisms and/or transport systems is required so that the host cells secrete the lipase into the fermentation medium. Without secretion, isolation of the lipase from the host cell, i.e. purification of the lipase from the cell mass, can take place, for example, by precipitation with ammonium sulfate or ethanol, or by chromatographic purification.

All of the circumstances listed above can be combined in a process to produce lipases as contemplated herein.

An additional subject as contemplated herein is an agent wherein it contains a lipase as described above. The agent is preferably a detergent or cleaning agent.

This subject matter as contemplated herein includes all feasible types of detergent or cleaning agent, both concentrates and undiluted agents to be applied, for use on a commercial scale, in the washing machine or for hand washing or cleaning. For example, this includes detergents for textiles, carpets or natural fibers for which the designation detergent is used. This also includes, for example, dishwashing detergent for dishwashing machine or manual dishwashing detergent or cleaners for hard surfaces, such as metal, glass, porcelain, ceramic, tiling, stone, painted surfaces, plastics, wood or leather for which the designation cleaning agent is used, i.e. in addition to manual and machine dishwashing detergents, for example, scouring agents, glass cleaner, WC fragrance rising aids, etc. The detergent and cleaning agents in the context of the disclosure also include washing aids which are added to the actual detergent in the manual or machine textile washing in order to achieve an enhanced effect. Furthermore, detergent and cleaning agents in the context of the disclosure also include textile pre-treatment and post-treatment agents, i.e. such agents with which the article to be washed comes into contact before the actual washing, for example, to dissolve stubborn soiling, as well as such agents which lend additional desirable properties, such as a pleasant feel, crease resistance or low static charge in a subsequent step to the actual textile washing. The last-mentioned agents include fabric softeners, among other things.

The detergent or cleaning agents as contemplated herein, which can be provided as a powdery solid, in post-compressed particle form and as homogeneous solutions or suspensions, can, in addition to a lipase, also include all known normal ingredients in such agents, wherein at least one additional ingredient is preferably included in the agent. The agents as contemplated herein can contain, in particular, surfactants, builders, peroxygen compounds or bleach activators. Moreover, they can contain water-miscible organic solvents, additional enzymes sequestering agents, electrolytes, pH regulators and/or additional auxiliary ingredients, such as optical lighteners, graying inhibitors, foam regulators and colorants and fragrances, as well as combinations thereof.

In particular, a combination of a lipase with one or multiple additional ingredients of the agent is beneficial, because such an agent in preferred variants as contemplated herein have enhanced cleaning performance with the resulting synergisms. In particular, combination of a lipase with a surfactant and/or a builder and/or a peroxygen compound and/or a bleach activator can achieve such a synergism.

Beneficial ingredients of agents as contemplated herein are disclosed in the international patent application WO2009/121725, starting on page 5, last paragraph and ending on page 13 after the second paragraph. Express reference is made to this disclosure and the content of the disclosure is taken into account into the present patent application.

An agent as contemplated herein beneficially contains the lipase in an amount of from about 2 μg to about 20 mg, preferably from about 5 μg to about 17.5 mg, more preferably from about 20 μg to about 15 mg and particularly from about 50 μg to about 10 mg per gram of agent. Moreover, the lipase contained in the agent, and/or additional ingredients of the agent can be surrounded by a substance that is impermeable for the enzyme at room temperature or with the absence of water, which is permeable to the enzyme under application conditions of the medium. One such embodiment as contemplated herein is thus wherein the lipase is surrounded by a substance that is impermeable for the enzyme at room temperature or with the absence of water. Furthermore, the detergent or cleaning agent itself can be packaged in a container, preferably a container that is permeable to air, from which it removed shortly before use or during the washing process.

In further embodiments as contemplated herein, the agent is wherein it (a) is provided in solid form, particularly as a pourable powder having a bulk weight of from about 300 g/l to about 1200 g/l, particularly from about 500 g/l to about 900 g/l, or
(b) is provided in paste-like or liquid form, and/or
(c) in provided in gel-like or dosage bag (pouch) form, and/or
(d) is provided as a single-component system, or
(e) is divided into multiple components.

These embodiments of the present disclosure comprise all solid, powdery, liquid, gel-like or pasty dosage forms of agents as contemplated herein, which can optionally consist of multiple phases and be provided in compressed or non-compressed form. The agent can be a pourable powder, in particular, with a bulk weight of from about 300 g/l to about 1200 g/l, particularly from about 500 g/l to about 900 g/l or from about 600 g/l to about 850 g/l. The solid dosage forms of the agent also include extrudates, granulates, tablets and pouches. Alternatively, the agent can also be liquid, gel-like or pasty, i.e. in the form of a non-hydrous liquid detergent or a non-hydrous paste or in the form of a hydrous liquid detergent or a hydrous paste. The agent can also be provided as a single-component system. Such agents consist of one phase. Alternatively, an agent can consist of multiple phases. One such agent is thus divided into multiple components.

Detergents or cleaning agents as contemplated herein can only contain one lipase. Alternatively, they can also contain additional hydrolytic enzymes or other enzymes in a concentration suitable for the effectiveness of the agent. An additional embodiment as contemplated herein, therefore, is an agent which comprises one or multiple additional enzymes. All enzymes which can develop catalytic activity in the agent can be used as additional enzymes, particularly protease, amylase, cellulase, hemicellulase, mannanase, tannase, xylanase, xanthanase, xyloglucanase, beta-glucosidase, pectinase, carrageenase, perhydrolase, oxidase, oxidoreductase or other lipases distinguishable from the lipases as contemplated herein, and mixtures thereof. Additional enzymes are beneficially contained in an amount of from about 1×10⁻⁸ to about 5 percent by weight relative to the active protein. Increasing preference is given to each additional enzyme contained in agents in an amount of from about 1×10⁻⁷ to about 3 wt. %, from about 0.00001 to about 1 wt. %, from about 0.00005 to about 0.5 wt. %, from about 0.0001 to about 0.1 wt. % and particularly from about 0.0001 to about 0.05 wt. % relative to the active protein. It is particularly preferred that the enzymes have a synergistic cleaning performance for specific stains, i.e. the enzymes contained in the agent composition support each other in the cleaning. It is particularly preferred that such synergism is provided between the lipase contained as contemplated herein and an additional enzyme of a agent, particularly between said lipase and an amylase and/or a protease and/or a mannanase and/or a cellulase and/or a pectinase. Synergistic effects can occur not only between different enzymes, but also between one or multiple enzymes and additional ingredients of the agent.

An additional subject as contemplated herein is a method for cleaning textiles or hard surfaces wherein an agent as contemplated herein is applied in at least one method step, or that a lipase as contemplated herein is catalytically activated in at least one method step, in particular, such that the lipase is used in an amount of from about 40 μg to about 4 g, preferably from about 50 μg to about 3 g, more preferably from about 100 μg to about 2 g and particularly from about 200 μg to about 1 g.

In various embodiments, the method described above is wherein the lipase is used at a temperature of from 0 to about 100° C., preferably from about 0 to about 60° C., more preferably from about 20 to about 40° C. and particularly about 34.8° C.

This includes manual and machine methods, where preference is given to machine methods. Methods for cleaning textiles are generally wherein different cleaning-active substances are applied on the object to be cleaned in multiple method steps and washed off after an exposure time or that the object to be cleaned is treated with a washing agent or a solution or dilution of said agent in another manner. The same applies for methods for cleaning of all materials other than textiles, particularly hard surfaces. All feasible washing or cleaning methods can be enhanced in at least one of the method steps with the use of a detergent or cleaning agent or a lipase as contemplated herein and then represent embodiments of the present disclosure. All circumstances, subject matter and embodiments which are described for lipases and the agents containing them are also applicable on this subject as contemplated herein. Therefore, express reference is hereby made to the disclosure in the relevant place with the notice that said disclosure also applies for the method described above.

Because lipases as contemplated herein already have natural hydrolytic activity and develop said activity in media which does not have any other cleaning force, such as pure buffers, a single or the only step of such a method can entail that a lipase as contemplated herein is brought into contact with the stain as a single active cleaning component, preferably in a buffer solution or in water. This is a further embodiment of this subject as contemplated herein.

Alternative embodiments of this subject as contemplated herein are methods for treatment of raw textile materials or for textile with which a lipase is active in at least one method step. Methods for raw textile materials, fibers or textiles with natural components are preferred, especially such methods for wool or silk.

Finally, the invention also comprises the use of the lipases described here in detergents or cleaning agents, such as those described above for (improved) removal of fatty stains, for example, on textiles or hard surfaces.

All circumstances, subject matter and embodiments which are described for lipase and the agents containing them are also applicable on this subject as contemplated herein. Therefore, express reference is hereby made to the disclosure in the relevant place with the notice that said disclosure also applies for the use described above.

### EXAMPLES

All molecular biological work steps follow standard methods such as those specified in the manual from Fritsch, Sambrook and Maniatis “Molecular cloning: a laboratory manual”, Cold Spring Harbour Laboratory Press, New York, 1989, or comparable reference works. Enzymes and kits were used according to the specifications of the respective manufacturers.

Overview of the Mutations:

| Variant | Sequence | | | | SEQ ID NO: |
|---|---|---|---|---|---|
| Variant 1 | P308S | | | | 2 |
| Variant 2 | S195R | S364C | | | 3 |
| Variant 3 | S195R | E335G | | | 4 |
| Variant 4 | Q294R | S364R | | | 5 |
| Variant 5 | T78S | | | | — |
| Variant 6 | E287V | | | | 6 |
| Variant 7 | N218I | I302T | | | 7 |
| Variant 8 | P292S | | | | 8 |
| Variant 9 | E335G | | | | 9 |
| Variant 10 | K142E | I149R | K204R | Q309L | 10 |
| Variant 11 | A207T | K235R | K339E | | — |

### Example 1

### Determination of the Thermal Stability of LipRO in a Detergent Matrix

The MTP expression in E.coli of a lipase of rhizopus oryzae with the amino acid sequence according to SEQ ID NO:1 and the variants with the AS sequences according to SEQ ID Nos. 2-10, were induced with the addition of IPTG after cultivation at 37° C. for 2 hours. Then the plates were cultivated at 30° C. for 4 hours. The cell pellets were re-suspended in a 150 μl lysozyme solution (1 mg×ml-1 in TEA buffer; 50 mM, pH 7.4). Then the plates were incubated at 37° C. and centrifuged at 900 rpm for 1 hour. Then the microtiter plates were centrifuged for 15 minutes (4000×g, 4° C.) and the clear excess material was used for the thermal stability test. 40 μl of clear excess material was transferred to a PCR microtiter plate and incubated together with Henkel matrix (1:200 in TEA buffer) for 30 minutes in a PCR thermocycler between 30 and 40° C. (1st step). Then the PCR microtiter plate was cooled on ice for 5 minutes and 40 μl of excess material was used for the pNP-butyrate-assay (2nd step).

The following detergent matrix was used (LSPA+):

| | wt. % of active | wt. % of active |
|---|---|---|
| | substance in the | substance in the |
| Chemical name | raw material | formulation |
| Demineralized water | 100 | Remaining |
| alkylbenzene sulfonic acid | 96 | 7.0 |
| Additional anionic surfactants | 70 | 9.0 |
| C12-C18 fatty acids Na salt | 30 | 4.0 |
| Nonionic surfactants | 100 | 7.0 |
| Phosphonates | 40 | 0.7 |
| Citric acid | 100 | 3.2 |
| NaOH | 50 | 3.0 |
| Defoamer | t.q. | 0.04 |
| 1,2-Propanediol | 100 | 5.7 |
| Preservative | 100 | 0.1 |
| Ethanol | 93 | 2.0 |
| Dye transfer inhibitor | 30 | 0.2 |

This matrix is provided with an additional 1% boric acid for the measurements with stabilizer.

### Activity Assay

For identification of variants with enhanced thermal stability, a microtiter plate-based assay was used with para-nitrophenol-butyrate (pNP butyrate) as a substrate. With enzymatic hydrolysis in the hydrous medium, para-nitrophenolate and butyrate were released and then para-nitrophenolate was detected at a wave length of 405 nm by means of absorption measurement. For the screening for thermal stability, the plates were incubated in parallel at 34.8° C. and at room temperature. Reaction conditions: 40 μl of clear excess material, which was obtained after cell lysis and centrifugation was added to the 10 μl matrix solution (1:200 dilution in 50 mM TEA buffer, pH 7.4) and mixed. Then a plate was incubated for 30 minutes at 34.8° C. in a PCR cycler, followed by a 5-minute incubation on ice. The room temperature plate was incubated for 35 minutes at room temperature. After the incubation, 40 μl of the reaction mixture was transferred to a new MPT. The enzyme reaction was initiated with addition of 60 μl of freshly produced pNP-butyrate solution (final concentration 1.5 mM) and the increase of the absorption was measured at a wave length of 405 nm with the Tecan Sunrise and XFLUOR software. The absorption was measured in accuracy mode over 60 cycles in 7-second intervals and the plates were shaken within the reading device for 2 minutes.

Production of 100 mM pNP-butyrate (base solution):
17.6 μl pNP-butyrate mixed 983 μl acetonitrile

Working pNP-butyrate concentrations:
7800 μl of 50 mM TEA buffer, pH 7.4 was mixed with 200 μl 100 mM pNP-butyrate

The dilution of the pNP butyrate base solution for the working concentration must take place before the measurement due to the high autohydrolysis.

The activity ratios of LipRO variants in the pNP-butyrate-MPT assay are listed below. The activity ratio was calculated by dividing (increase/min of activity value) at 34.8° C. by (increase/min of activity value) at room temperature.

| Variant (SEQ ID NO) | Activity ratio (increase/min of activity value) |
|---|---|
| LipRO WT (1) | 4.3 |
| Variant 1 (2) | 16.9 |
| Variant 2 (3) | 9.8 |
| Variant 3 (4) | 10.1 |
| Variant 4 (5) | 13.8 |
| Variant 5 | 4.1 |
| Variant 6 (6) | 73.8 |
| Variant 7 (7) | 46.2 |
| Variant 8 (8) | 9.5 |
| Variant 9 (9) | 8.8 |
| Variant 10 (10) | 14.0 |
| Variant 11 | 1.8 |

Variants 5 and 11 are comparison examples in which the amino acid substitution did not cause any improvement in stability.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A lipase comprising an amino acid sequence having a sequence that is at least about 70% identical to the amino acid sequence specified in SEQ ID NO:1 over the entire length and an amino acid substitution of at least one of the positions K142, I149, S195, K204, N218, E287, P292, Q294, I302, P308, Q309, E335 or S364, corresponding to the numbering in accordance with SEQ ID NO:1.

2. Lipase according to claim 1, wherein the at least one amino acid substitution is selected from the group of K142E, I149R, S195R, K204R, N218I, E287V, P292S, Q294R, I302T, P308S, Q309L, E335G, S364C and S364R, corresponding to the numbering in accordance with SEQ ID NO:1.

3. Lipase according to claim 1, wherein the lipase has one of the following amino acid substitutions:
(i) P308S;
(ii) S195R and S364C;
(iii) S195R and E335G;
(iv) Q294R and S364R;
(v) E287;
(vi) N218I and I302T;
(vii) P292S;
(viii) E335G; or
(iv) K142E, I149R, K204R and Q309L.

4. Lipase, wherein
(a) it can be obtained from a lipase according to claim 1 as an original molecule by employing single or multiple conservative amino acid substitution, wherein the lipase has at least one of the amino acid substitutions K142E, I149R, S195R, K204R, N218I, E287V, P292S, Q294R, I302T, P308S, Q309L, E335G, S364C or S364R, corresponding to the positions 142, 149, 195, 204, 218, 287, 292, 294, 302, 308, 309, 335 and 364 according to SEQ ID NO:1; and/or
(b) it can be obtained from a lipase according to claim 1 by fragmenting, deletion-, insertion- or substitution mutagenesis and comprises an amino acid sequence which matches the original molecule over a length of at least about 50 linked amino acids, wherein the lipase comprises at least one of the amino acid substitutions K142E, I149R, S195R, K204R, N218I, E287V, P292S, Q294R, I302T, P308S, Q309L, E335G, S364C or S364R corresponding to the positions 142, 149, 195, 204, 218, 287, 292, 294, 302, 308, 309, 335 and 364 according SEQ ID NO:1.

5. A method for production of a lipase comprising the substitution of an amino acid in at least one of the positions corresponding to position 142, 149, 195, 204, 218, 287, 292, 294, 302, 308, 309, 335 and 364 in SEQ ID NO:1, in an original lipase having a sequence that is at least about 70% identical to the amino acid sequence specified in SEQ ID NO:1 over the entire length such that the lipase comprises the amino acid substitution K142E, I149R, S195R, K204R, N218I, E287V, P292S, Q294R, I302T, P308S, Q309L, E335G, S364C or S364R in at least one position.

6. Method according to claim 5 additionally comprising one or both of the following method steps:
a) Introduction of a single or multiple conservative amino acid substitution, wherein the lipase comprises at least one of the amino acid substitutions K142E, I149R, S195R, K204R, N218I, E287V, P292S, Q294R, I302T, P308S, Q309L, E335G, S364C or S364R, corresponding to the positions 142, 149, 195, 204, 218, 287, 292, 294, 302, 308, 309, 335 and 364 according to SEQ ID NO:1;
b) Change of the amino acid sequence by fragmenting, deletion-, insertion- or substitution mutagenesis such that the lipase comprises an amino acid sequence which matches the original molecule over a length of at least about 50 linked amino acids, wherein the lipase comprises at least one of the amino acid substitutions K142E, I149R, S195R, K204R, N218I, E287V, P292S, Q294R, I302T, P308S, Q309L, E335G, S364C or S364R corresponding to the positions 142, 149, 195, 204, 218, 287, 292, 294, 302, 308, 309, 335 and 364 according SEQ ID NO:1.

7. (canceled)

8. (canceled)

9. (canceled)

10. (canceled)

11. An agent, wherein the agent comprises at least one lipase according to claim 1.

12. (canceled)

13. Lipase according to claim 1, wherein the lipase is utilized in a detergent or cleaning agent for removal of fatty stains.

14. Agent according to claim 11, wherein the agent is further defined as a detergent or cleaning agent.

15. Lipase according to claim 1, wherein lipase comprises the amino acid substitution P308S, corresponding to the numbering in accordance with SEQ ID NO:1.

16. Lipase according to claim 1, wherein lipase comprises the amino acid substitutions S195R and S364C, corresponding to the numbering in accordance with SEQ ID NO:1.

17. Lipase according to claim 1, wherein lipase comprises the amino acid substitutions S195R and E335G, corresponding to the numbering in accordance with SEQ ID NO:1.

18. Lipase according to claim 1, wherein lipase comprises the amino acid substitutions Q294R and S364R, corresponding to the numbering in accordance with SEQ ID NO:1.

19. Lipase according to claim 1, wherein lipase comprises the amino acid substitution E287P, corresponding to the numbering in accordance with SEQ ID NO:1.

20. Lipase according to claim 1, wherein lipase comprises the amino acid substitutions N218I and I302T, corresponding to the numbering in accordance with SEQ ID NO:1.

21. Lipase according to claim 1, wherein lipase comprises the amino acid substitution P292S, corresponding to the numbering in accordance with SEQ ID NO:1.

22. Lipase according to claim 1, wherein lipase comprises the amino acid substitution E335G, corresponding to the numbering in accordance with SEQ ID NO:1.

23. Lipase according to claim 1, wherein lipase comprises the amino acid substitution K142E, I149R, K204R and Q309L, corresponding to the numbering in accordance with SEQ ID NO:1.
